(19) United States
(12) Patent Application Publication
(10) Pub. No.: US 2014/0243383 A1
(43) Pub. Date: Aug. 28, 2014

(54) PHARMACEUTICAL COMPOSITIONS OF SILODOSIN

(71) Applicants: Sunilendu Bhushan Roy, Gujarat (IN); Sushrut Krishnaji Kulkarni, Gujarat (IN); Ajay Kumar Handa, Gujarat (IN); Doss Pothuvan Uma, Gujarat (IN)

(72) Inventors: Sunilendu Bhushan Roy, Gujarat (IN); Sushrut Krishnaji Kulkarni, Gujarat (IN); Ajay Kumar Handa, Gujarat (IN); Doss Pothuvan Uma, Gujarat (IN)

(73) Assignee: CADILA HEALTHCARE LIMITED, Ahmedabad (IN)

(21) Appl. No.: 14/238,777

(22) PCT Filed: Aug. 24, 2012
(86) PCT No.: PCT/IN2012/000565

§ 371 Date: Apr. 16, 2014

(30) Foreign Application Priority Data
Aug. 24, 2011 (IN) 2375/MUM/2011

(51) Int. Cl. A61K 31/404

(52) U.S. Cl. CPC A61K 31/404
USPC 514/415

(57) ABSTRACT

The present invention discloses a stable pharmaceutical composition comprising: (a) plurality of granules comprising silodosin or salts thereof and one or more pharmaceutical excipients; and (b) an extragranular portion comprising one or more lubricants and optionally one or more surfactants, wherein the granules are free of partially pregelatinized starch.

### FIELD OF THE INVENTION

The present invention relates to pharmaceutical compositions of silodosin or salts thereof. In particular, the invention relates to stable pharmaceutical compositions of silodosin or salts thereof with one or more pharmaceutical excipients. The invention also relates to processes for the preparation of such compositions and use thereof for treatment of benign prostatic hyperplasia.

### BACKGROUND OF THE INVENTION

Silodosin is an α-adrenergic antagonist that has high selectivity for the α_{1A} receptor relative to α_{1B} and α_{1D} receptors. Silodosin is used for the treatment of dysuria, has selective suppressing activities on the contraction of urethra smooth muscles, and is an extremely useful compound as a medicament for treating dysuria without causing strong hypotensive activities or orthostatic hypotension.

Chemically, silodosin is an indoline derivative having the molecular formula C₂₅H₃₂F₃N₃O₄ and the following chemical structure:

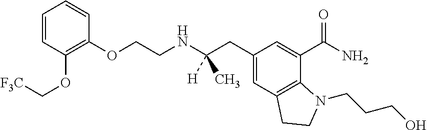 [SMILES: C[C@H](Cc1cc2c(c(C(N)=O)c1)N(CCCO)CC2)NCCOc1ccccc1OCC(F)(F)F]

Silodosin is approved in USA for 4 mg twice daily dosing and 8 mg once daily dosing to treat symptoms associated with benign prostatic hyperplasia (“BPH”) and is marketed by Watson Labs under the tradename Rapaflo®.

BPH is non-cancerous growth of the prostate gland, which most typically occurs in middle-aged and elderly men. The symptoms of BPH include, for example, weak or intermittent urinary stream (flow rate), straining when urinating, a hesitation before urine flow starts, a sense that the bladder has not emptied completely, and dribbling at the end of urination or leakage afterward.

Twice daily dosage regimen of silodosin is, however, less preferred over once-daily dosing regimens because the later is generally more convenient and increases patient compliance.

Silodosin is known to possess incompatibilities with several pharmaceutical excipients and that yields degradation products of silodosin. For example, a most popularly used filler, lactose when used as in formulating the tablets of silodosin; the resulting tablets exhibits undesirable dissolution properties and unsatisfactory hardness. Moreover, silodosin has a potent adhesive property, and in the case of preparing a tablet or capsule, use of a lubricant is inevitable. On the contrary, the addition of such lubricants causes the problem of delaying in dissolution time. Furthermore, silodosin is relatively unstable against a light exposure.

Silodosin has also potent adhesive and electrostatic properties. Particularly, in cases where formulations are prepared by a dry process, electrostatic charges are generated by physical irritations caused through processes such as pulverization, agitation, blending, granulation and the like, which in turn cause a decrease in fluidity of pulverized, blended or granulated materials, worsen handling properties, and decrease precision for content uniformity of an active ingredient.

The synthesis of silodosin is described in U.S. Pat. No. 5,387,603.

U.S. Patent Publication No. U.S. 2009/0143404 discloses a method for treating a patient having symptoms associated with benign prostatic hyperplasia comprising administering once daily to said patient a pharmaceutical composition comprising an effective amount of silodosin or a pharmaceutically acceptable salt thereof. The pharmaceutical composition typically comprises silodosin or salt thereof and at least one pharmaceutically acceptable excipient.

U.S. Patent Publication No. U.S. 2006/0018959 discloses a capsule comprising a granule containing silodosin, D-mannitol and two types of partially gelatinized starches and the extragranular part contains a lubricant and sodium lauryl sulfate.

Despite of the suggested methods in the art, it is extremely difficult to prepare practically a stable and usable pharmaceutical composition comprising, as an active ingredient, silodosin or salt thereof by conventional formulation methods which can achieve precise content uniformity.

The prior art references emphasize on using partially pregelatinized starches to improve the dissolution profile of silodosin in water and there is no disclosure or teaching/suggestion in the art about how to develop stable formulations of silodosin without employing partially pregelatinized starch and which composition also ought to exhibit dissolution profile suitable for once daily administration of said formulation.

Thus, there still remains a need for alternative pharmaceutical formulations comprising silodosin in order to achieve improved dissolution profile suitable for once daily administration and also possess excellent storage stability.

### SUMMARY OF THE INVENTION

In one general aspect there is provided a stable pharmaceutical composition comprising—
(a) plurality of granules comprising silodosin or salts thereof and one or more pharmaceutical excipient/s; and
(b) an extragranular portion comprising one or more lubricant/s and optionally one or more surfactant/s,
wherein said granules are free of partially pregelatinized starch.

In another general aspect there is provided a stable pharmaceutical composition comprising—
(a) plurality of granules comprising silodosin or salts thereof, one or more surfactant/s and one or more pharmaceutical excipient/s; and
(b) an extragranular portion comprising one or more lubricant/s and optionally one or more surfactant/s,
wherein said granules are free of partially pregelatinized starch.

In another general aspect there is provided a stable pharmaceutical composition comprising—
(a) plurality of granules comprising silodosin or salts thereof and one or more pharmaceutical excipient/s; and
(b) an extragranular portion comprising one or more lubricant/s and optionally one or more surfactant/s,
wherein said granules are free of lactose and partially pregelatinized starch.

In another general aspect there is provided a stable pharmaceutical composition comprising—
(a) plurality of granules comprising, silodosin or salts thereof and one or more pharmaceutical excipient/s; and
(b) an extragranular portion comprising one or more lubricant/s and optionally one or more surfactant/s,
wherein said granules are free of partially pregelatinized starch and said composition exhibits a dissolution profile of silodosin or salt thereof in water such that at least 85% silodosin is released in not more than 60 minutes.

In another general aspect there is provided a stable pharmaceutical composition comprising—
(a) plurality of granules comprising silodosin or salts thereof and one or more pharmaceutical excipient/s; and
(b) an extragranular portion comprising one or more lubricant/s and optionally one or more surfactant/s,
wherein said granules are free of partially pregelatinized starch and said composition exhibits a dissolution profile of silodosin or salt thereof in water such that at least 80% silodosin is released in not more than 15 minutes.

In another general aspect there is provided a stable pharmaceutical composition comprising—
(a) plurality of granules comprising silodosin or salts thereof, one or more surfactant/s, one or more binder/s, one or more filler/s and one or more pharmaceutical excipients;
(b) an extragranular portion comprising one or more lubricant/s and optionally one or more surfactant/s,
wherein said granules are free of partially pregelatinized starch.

In another general aspect there is provided a stable pharmaceutical composition comprising—
(a) plurality of granules comprising silodosin or salts thereof, a polyoxyethylene polyoxypropylene copolymer, mannitol, optionally polyvinyl pyrrolidone, and one or more pharmaceutical excipients;
(b) an extragranular portion comprising magnesium stearate and optionally sodium lauryl sulfate,
wherein said granules are free of partially pregelatinized starch.

In another general aspect there is provided a stable pharmaceutical composition comprising—
(a) plurality of granules comprising silodosin or salts thereof, one or more surfactant/s and one or more pharmaceutical excipient/s; and
(b) an extragranular portion comprising one or more lubricant/s and optionally one or more surfactant/s,
wherein the ratio of amount of silodosin or salt thereof to the total amount of surfactant in the composition ranges from about 1:0.5 to about 0.5:1, and said granules are free of partially pregelatinized starch.

In another general aspect there is provided a stable pharmaceutical composition comprising—
(a) plurality of granules comprising silodosin or salts thereof, one or more surfactant/s and one or more pharmaceutical excipient/s; and
(b) an extragranular portion comprising one or more lubricant/s and optionally one or more surfactant/s,
wherein the ratio of amount of silodosin or salt thereof to the amount of surfactant in the granules ranges from about 2:1 to about 1:1, and said granules are free of partially pregelatinized starch.

Embodiments of the stable pharmaceutical composition may include one or more of the following features. For example, the pharmaceutical composition may further include one or more pharmaceutically acceptable excipients. The pharmaceutically acceptable excipients may include one or more binders, fillers, lubricants, disintegrants, glidants and the like.

In another general aspect there is provided a stable pharmaceutical composition comprising—
(a) plurality of granules comprising silodosin or salts thereof and one or more pharmaceutical excipient/s; and
(b) an extragranular portion comprising one or more lubricant/s and optionally one or more surfactant/s,
wherein said granules are free of partially pregelatinized starch and said composition retains at least 80% of the potency of silodosin or salts thereof in the pharmaceutical composition after storage at 40° C. and 75% relative humidity for three months.

Embodiments of the stable pharmaceutical composition may include one or more of the following features. For example, the pharmaceutical composition may further include one or more pharmaceutically acceptable excipients. The pharmaceutically acceptable excipients may include one or more binders, fillers, lubricants, disintegrants, glidants and the like.

In another general aspect, there is provided a process of preparing a stable pharmaceutical composition, which process comprises of—
(a) preparing plurality of granules comprising silodosin or salts thereof and one or more pharmaceutical excipient/s;
(b) mixing the granules prepared in step (a) with one or more lubricant/s and optionally one or more surfactant/s to form a blend; and
(c) formulating the blend into a single unit dosage form,
wherein said granules are free of partially pregelatinized starch.

Embodiments of the stable pharmaceutical composition may include one or more of the following features. For example, the pharmaceutical composition may further include one or more pharmaceutically acceptable excipients. The pharmaceutically acceptable excipients may include one or more binders, fillers, lubricants, disintegrants, glidants and the like.

In another general aspect there is provided a method of treating benign prostatic hyperplasia in patients, wherein the method comprises administering a stable pharmaceutical composition comprising—
(a) plurality of granules comprising silodosin or salts thereof and one or more pharmaceutical excipient/s; and
(b) an extragranular portion comprising one or more lubricant/s and optionally one or more surfactant/s,
wherein said granules are free of partially pregelatinized starch.

Embodiments of the stable pharmaceutical composition may include one or more of the following features. For example, the pharmaceutical composition may further include one or more pharmaceutically acceptable excipients. The pharmaceutically acceptable excipients may include one or more binders, fillers, lubricants, disintegrants, glidants and the like.

In another general aspect there is provided a method of treating benign prostatic hyperplasia comprising administering a stable pharmaceutical composition of silodosin as per the invention.

The details of one or more embodiments of the present invention are set forth in the description below. Other features, objects and advantages of the invention will be apparent from the description.

### DETAILED DESCRIPTION OF THE INVENTION

The inventors of the present invention have surprisingly found that formulations of silodosin can be prepared without using any partially pregelatinized starch and such formulations has a high precision of content uniformity, excellent stability and an in vitro (dissolution) profile suitable for once daily administration.

The inventors have developed stable pharmaceutical compositions of silodosin by careful selection of pharmaceutical excipients with their optimum concentrations. Such formulation was surprisingly found to remain stable over the storage period, in particular, retaining at least 80% of the potency of silodosin or salts thereof in the pharmaceutical composition after storage at 40° C. and 75% relative humidity for three months.

Embodiments of the present invention relate to stable pharmaceutical composition comprising—
(a) plurality of granules comprising silodosin or salts thereof and one or more pharmaceutical excipient/s; and
(b) an extragranular portion comprising one or more lubricant/s and optionally one or more surfactant/s,
wherein said granules are free of partially pregelatinized starch.

As used herein, the term ‘silodosin’ is used in broad sense to include not only the silodosin per se but also its pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs and pharmaceutically acceptable prodrugs thereof, and also its various crystalline and amorphous forms.

As used herein, the term ‘granule’ is used to refer any particulate form of matter such as particles, cores, pellets, agglomerates, and the like.

The pharmaceutical composition is desired to show good dissolution properties in the stomach except for cases where the pharmaceutical are enteric coated formulations due to their unstable properties in acidic conditions. Since silodosin is stable in acidic conditions, compositions of silodosin are desired to show good dissolution properties in the first fluid, which is corresponding to gastric juice, in a dissolution test. Accordingly, in the pharmaceutical composition in accordance with the present invention, about 85% of silodosin or salt thereof is dissolved in not more than 60 minutes in a dissolution test using the first fluid as in cases where the dissolution test is carried out using water as medium and USP type II dissolution apparatus (paddle speed of 50 rpm). In an embodiment, about 85% of silodosin or salt thereof is dissolved in not more than 30 minutes, and about 80% of silodosin or salt thereof is dissolved in not more than 15 minutes.

In an embodiment, the granules of the pharmaceutical composition comprise silodosin or salt thereof and one or more surfactants. The inventors have, further surprisingly found that macrogol (polyethyleneglycol), polyoxyethylene polyoxypropylene glycol (available commercially under trade name of Poloxamers and pluroics®) when used as surfactants, preferably in the granules, it may contribute in exhibiting excellent storage stability and dissolution profile of silodosin desired for once daily administration.

In a further embodiment, the extragranular portion of the pharmaceutical composition comprising silodosin or salt thereof is free of any surfactant.

In a further embodiment, the granules of the pharmaceutical composition comprising silodosin or salt thereof are free of lactose.

In a further embodiment, the granules of the pharmaceutical composition comprising silodosin or salt thereof are free of lactose and partially gelatinized starch.

Further, in contrary to the teaching of the prior art, the inventors have surprisingly found that when celluloses such as hydroxypropyl methylcellulose and hydroxypropyl cellulose are used as a binder, it do not cause incompatibility with silodosin.

Thus, in a further embodiment, the pharmaceutical composition comprises—
(a) plurality of granules comprising silodosin or salts thereof, one or more surfactant/s, one or more binder/s and one or more pharmaceutical excipient/s; and
(b) an extragranular portion comprising one or more lubricant/s and optionally one or more surfactant/s.

In a still further embodiment, ratio of amount of silodosin or salt thereof and the total amount of surfactant in the pharmaceutical ranges from about 1:0.5 to about 0.5:1, and the ratio of amount of silodosin or salt thereof and the total amount of surfactant in the granules ranges from about 2:1 to about 1:1.

The pharmaceutical composition of the present invention can be formulated into tablets, minitablets, capsule or sachet. The pharmaceutical composition comprises plurality of granules mixed or compressed with one or more lubricants, optionally with one or more lubricants.

In an embodiment the pharmaceutical composition is provided in the form of capsule comprising plurality of granules comprising silodosin or salt thereof, and an extragranular portion comprising one or more pharmaceutical excipients, wherein the composition is free of partially pregelatinized starch.

In a further embodiment the pharmaceutical compositions are in multiple unit form, such as pellets or granules. Alternatively said multiple units can be processed further into solid dosage forms such as tablet, minitablets or said multiple units can be filled into capsules or sachets.

In a further embodiment multiple units/core comprise non-pareil seeds or sugar spheres or neutral excipients or water soluble/insoluble and/or swellable excipients coated with one or more drug layers comprising silodosin or salts thereof with one or more water-insoluble excipients. The multiple unit cores may be further coated with one or more layers of pharmaceutical excipients.

In a further embodiment the pharmaceutical composition or granules therein may optionally have functional as well non-function coating. The functional coatings may include controlled-release and/or delayed release coating and non-functional coating may include seal coatings and/or elegant coatings.

Furthermore, the stability of the pharmaceutical of the present invention against

a light exposure can be improved by careful handling, storing under a light-resistant packaging.

In an embodiment, the pharmaceutical composition or the functional/non-function coating of the composition may comprise titanium oxide, a light-shielding material. A highly photo-stable pharmaceutical composition can be prepared by using capsules containing titanium oxide or coating agents containing titanium oxide.

The pharmaceutical composition of silodosin or salts thereof can be prepared by any suitable method known in the art such as direct compression, dry or wet granulation, fluidized bed granulation, melt extrusion, melt granulation, spray coating, freeze drying, spray drying and solution evaporation.

In an embodiment the process of preparing the pharmaceutical composition comprises admixing, granulating and/or coating silodosin or salts thereof with one or more pharmaceutical excipients.

In a further embodiment the pharmaceutical composition may be prepared by admixing, granulating and/or coating silodosin or salts thereof with a binder together with one or more pharmaceutical excipient; admixing the resulting granules with one or more lubricants and optionally with one or more surfactants; and formulating (compressing or filling) the resulting admixture into a suitable dosage form.

In a further embodiment the process of preparing a stable pharmaceutical composition comprise steps of—
(a) preparing plurality of granules comprising silodosin or salts thereof and one or more pharmaceutical excipient/s;
(b) mixing the granules prepared in step (a) with one or more lubricant/s and optionally one or more surfactant/s to form a blend; and
(c) formulating the blend into a single unit dosage form.

The compositions may be formulated into dosage forms suitable for oral administration by precisely using pharmaceutical excipients such as diluents, fillers, binder, disintegrant, lubricant, glidants, surfactants, sweeteners, flavors etc or other additives which will render desired properties to resulting dosage form in accordance with the present invention.

Suitable diluents or bulking agents, which includes, but are not limited to, saccharides, including monosaccharides, disaccharides, polysaccharides and sugar alcohols such as arabinose, dextrose, sucrose, fructose, maltose, mannitol, erythritol, sorbitol, xylitol lactitol, and other bulking agents such as powdered cellulose, microcrystalline cellulose, sugar and derivatives thereof. The formulation may incorporate one or more of the above bulking agents, preferably, lactose & microcrystalline cellulose forms the bulking agent. The amount of the diluents or bulking agent is preferably in the range of 15% to 70% by weight of the composition.

Suitable binders, which includes, but are not limited to, acacia mucilage, alginic acid, carbomer, carboxymethylcellulose calcium, carboxymethylcellulose sodium, microcrystalline cellulose, powdered cellulose, ethyl cellulose, gelatin, liquid glucose, guar gum, xanthan gum, hydroxyethyl cellulose, hydroxypropyl cellulose, low-substituted hydroxypropyl cellulose (L-HPC), hydroxypropylmethyl cellulose, maltodextrin, methylcellulose, polydextrose, polyvinyl pyrrolidone (e.g. Povidone K30), polyethylene oxide, sodium alginate, corn starch, starch paste, and sucrose.

Suitable disintegrants, which includes, but are not limited to, croscarmellose sodium, crospovidone, sodium starch glycolate, corn starch, potato starch, maize starch and modified starches, calcium silicates, low substituted hydroxy-propylcellulose. The amount of disintegrating agent is preferably in the range of 5% to 35% by weight of the composition.

Suitable surfactants which may include, but are not limited to, anionic, cationic, non-ionic or amphoteric surfactants or those known to the person skilled in the art. The amount of surfactant present in the pharmaceutical composition of silodosin, or salt thereof ranges from about 0.5% to about 25% by total weight of the composition.

Suitable lubricants and/or glidants, which may include, but are not limited to, stearic acid and its derivatives or esters like sodium stearate, magnesium stearate and calcium stearate and the corresponding esters such as sodium stearyl fumarate; talc and colloidal silicon dioxide respectively. The amount of lubricant and/or glidant is preferably in the range of 0.25% to 5% by weight of the composition.

The invention further provides a method of treating benign prostatic hyperplasia in patients, wherein the method comprises administering the stable pharmaceutical composition in accordance with the present invention as substantially described hereinbefore.

In the context of the present invention, “Bioequivalency” is determined by a 90% Confidence Interval (CI) of between 0.80 and 1.25 for both Cₘₐₓ and AUC under USFDA regulatory guidelines, or a 90% CI for AUC of between 0.80 to 1.25 and a 90% CI for Cₘₐₓ of between 0.70 to 1.43 under the European regulatory guidelines (EMEA).

The term “confidence interval, (CI)” as used herein refers to the plain meaning known to one of ordinary skill in the art. The confidence interval refers to a statistical range with a specified probability that a given parameter lies within the range.

The term “covariance, (CV)” as used herein refers to the plain meaning known to one of ordinary skill in the art. It is a statistical measure of the variance of two random variables that are observed or measured in the same mean time period. This measure is equal to the product of the deviations of corresponding values of the two variables from their respective means.

The bioequivalence studies were carried out between Rapaflo® (reference) and compositions of the invention (test) in fasted and fed state. The study was monitored in terms of Cₘₐₓ, AUC, Tₘₐₓ achieved with the test products and the reference product (Rapaflo®).

At 90% confidence interval; area under the concentration time curve (AUCₒ₋ₜ and/or AUC_{o-inf}) and maximum plasma concentration (Cₘₐₓ) values of composition of the invention lies between 0.8 and 1.2 as compared to that obtained by silodosin formulation marketed under the trade name Rapaflo®.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### EXAMPLE 1

TABLE 1

| Sr. | | Qty |
|---|---|---|
| No. | Ingredient | (% w/w) |
| 1 | Silodosin (Particle size Below 5 micron) | 2-3% |
| 2 | Corn starch | 7-16% |
| 3 | L-HPC | 2-5% |
| 4 | Mannitol SD 200 | 50-80% |
| 5 | Poloxamer 407 (dry mix) | 2-5% |
| 6 | Poloxamer 407 (In water) | 2-5% |
| 7 | Water | Q.S. |
| 8 | Sodium Lauryl Sulphate | 1-3% |
| 9 | Magnesium stearate | 0.1-1.5% |
| | Total | 100.00 |

Procedure: Mixture of silodosin, corn starch, L-HPC, mannitol SD 200 and Poloxamer 407 (dry mix) was granulated using aqueous dispersion of Poloxamer 407 (in water). The granules were dried and mixed with sodium lauryl sulfate and magnesium stearate. The resulting blend was then filled into capsule.

The stability study of this formulation was conducted at 40° C./75 % R.H. over the period of 3 months.

The amount of the impurities measured in the formulation after the storage period indicates that the formulation of the invention is stable under stress conditions.

### STABILITY DATA OF EXAMPLE 1

| | | Time in Months | | | |
|---|---|---|---|---|---|
| Test | Specification with limits | Initial | 1 | 2 | 3 |
| Related | Impurity 2 - NMT 0.62% | 0.07 | 0.08 | 0.07 | 0.08 |
| substance | Impurity 4 - NMT 0.62% | BQL | BQL | BQL | BQL |
| | Impurity 5 - NMT 0.62% | 0.04 | 0.04 | 0.04 | 0.03 |
| | Unknown - NMT 0.25% | BQL | BQL | BQL | BQL |
| | Total - NMT 1.5% | 0.11 | 0.12 | 0.11 | 0.11 |

### Impurity 2:

(R)-1-(3-Hydroxypropyl)-5-(2-(2-(2-(2,2,2-trifluroethoxy)phenoxy)ethylamino) propyl)1H-indole-7-carboxamide

### Impurity 4:

2,3-dihydro-1-(3-hydroxypropyl)-5-[(2R)-2-[[2-(2,2,2-trifluroethoxy)phenoxy) Ethyl]amino]propyl]1H-indole-7-carboxamide (N-oxide)

### Impurity 5:

(R)-1-(3-Hydroxypropyl)-5-(2-(2-(2-(2,2,2-trifluroethoxy)phenoxy)ethylamino) propyl)indoline-7-carboxylic acid.

### EXAMPLE 2

TABLE 2

| | | Qty |
|---|---|---|
| Sr. No. | Ingredient | (% w/w) |
| 1 | Silodosin (Particle size Below 5 micron) | 2-3% |
| 2 | Corn starch | 12-15% |
| 3 | L-HPC | 2.5-5% |
| 4 | Mannitol SD 200 | 50-80% |
| 5 | Povidone K 30 (dry mix) | 0.5-2.5% |
| 6 | Povidone K 30 (In water) | 0.5-2.5% |
| 7 | Poloxamer 407 (dry mix) | 0.5-2.5% |
| 8 | Poloxamer 407 (In water) | 0.5-2.5% |
| 9 | Water | Q.S. |
| 10 | Sodium Lauryl Sulphate | 1-3% |
| 11 | Magnesium stearate | 0.1-1.5% |
| | Total | 100.00 |

Procedure: Mixture of silodosin, corn starch, L-HPC, mannitol SD 200, Povidone K 30 (dry mix) and Poloxamer 407 (dry mix) was granulated using aqueous dispersion of Povodone K 30 (in water) and Poloxamer 407 (in water). The granules were dried and mixed with sodium lauryl sulfate and magnesium stearate. The resulting blend was then filled into capsule.

1. A stable pharmaceutical composition comprising:
(a) plurality of granules comprising silodosin or salts thereof and one or more pharmaceutical excipients; and
(b) an extragranular portion comprising one or more lubricants and optionally one or more surfactants,
wherein the granules are free of partially pregelatinized starch.

2. A stable pharmaceutical composition according to claim 1, wherein the plurality of granules further comprise one or more surfactants.

3. The stable pharmaceutical composition as claimed in claim 1, wherein the granules are free of lactose.

4. The stable pharmaceutical composition as claimed in claim 1, wherein the composition exhibits a dissolution profile of silodosin or a salt thereof in water such that at least 85% silodosin is released in not more than 60 minutes.

5. A stable pharmaceutical composition comprising:
(a) plurality of granules comprising silodosin or salts thereof, a polyoxyethylene polyoxy propylene copolymer, mannitol, optionally polyvinyl pyrrolidone, and one or more pharmaceutical excipients; and
(b) an extragranular portion comprising magnesium stearate and optionally sodium lauryl sulfates,
wherein said granules are free of partially pregelatinized starch.

6. The stable pharmaceutical composition as claimed in claim 1, wherein the ratio of amount of silodosin or salt thereof to the total amount of surfactant in the composition ranges from about 1:5 to about 0.5:1.

7. The stable pharmaceutical composition as claimed in claim 1, wherein the ratio of amount of silodosin or salt thereof to the amount of surfactant in the granules ranges from about 2:1 to about 1:1.

8. The stable pharmaceutical composition as claimed in claim 1, wherein the composition retains at least 80% of the potency of silodosin or salts thereof in the pharmaceutical composition after storage at 40° C. and 75% relative humidity for three months.

9. The stable composition as claimed in claim 1, wherein said composition contains less than 0.62% w/w of impurity 2 relative to total weight of silodosin or salt thereof after storage for three months at 40° C. and 75% relative humidity.

10. The stable composition as claimed in claim 1, wherein said composition contains less than 0.62% w/w of impurity 4 relative to total weight of silodosin or salt thereof after storage for three months at 40° C. and 75% relative humidity.

11. The stable composition as claimed in claim 1, wherein said composition contains less than 0.62% w/w of impurity 5 relative to total weight of silodosin or salt thereof after storage for three months at 40° C. and 75% relative humidity.

12. The stable composition according to claim 1, wherein the composition comprises one or more pharmaceutically acceptable excipients selected from diluents, fillers, binders, disintegrants, lubricants, glidants, surfactants, sweeteners, and flavors.

13. A process for preparing a stable pharmaceutical composition as claimed in claim 1, which comprises:
(a) preparing plurality of granules comprising silodosin or salts thereof and one or more pharmaceutical excipients;
(b) mixing the granules prepared in step (a) with one or more lubricants and optionally one or more surfactants to form a blend; and
(c) formulating the blend into a single unit dosage form,
wherein said granules are free of partially pregelatinized starch.

14. The process of preparing a stable pharmaceutical composition as claimed in claim 2 which comprises:
(a) preparing plurality of granules comprising silodosin or salts thereof one or more surfactants and one or more pharmaceutical excipients;
(b) mixing the granules prepared in step (a) with one or more lubricants and optionally one or more surfactants to form a blend; and
(c) formulating the blend into a single unit dosage form,
wherein said granules are free of partially pregelatinized starch.

15. The stable pharmaceutical composition as claimed in claim 1, wherein the composition exhibits no significant difference in rate and/or extent of absorption of silodosin as compared to commercially marketed formulation of silodosin marketed under the trade name Rapaflo®.

16. (canceled)

17. A method of treating benign prostatic hyperplasia comprising administering to a subject in need thereof a stable pharmaceutical composition as claim in claim 1.

18. A method of treating benign prostatic hyperplasia comprising administering to a subject in need thereof a stable pharmaceutical composition as claimed in claim 2.

19. A method of treating benign prostatic hyperplasia comprising administering to a subject in need thereof a stable pharmaceutical composition as claimed in claim 5.
